(19) United States
(12) Patent Application Publication
(10) Pub. No.: US 2009/0182255 A1
(43) Pub. Date: Jul. 16, 2009

(54) Device and Method for Treating Ear Injuries

(76) Inventors: Duff M. Hay, Sandwich, IL (US); Benjamin D. Hay, Fithian, IL (US)

Correspondence Address: THE LAW OFFICE OF RANDALL T. ERICKSON, P.C., 1749 S. NAPERVILLE ROAD, SUITE 202, WHEATON, IL, 60187, US

(21) Appl. No.: 12/410,225

(22) Filed: Mar. 24, 2009

Related U.S. Application Data
(63) Continuation of application No. 11/544,721, filed on Oct. 10, 2006.

(51) Int. Cl. A61F 13/00

(52) U.S. Cl. 602/53

(57) ABSTRACT

An improved non-invasive ear compression dressing or splinting device is provided for prevention, treatment and recurrence of injuries to the outer ear such as auricular hematoma. The device includes a structure including a pair of first and second pads, and a pressure applying assembly. The pads are assembled in pairs in opposing, facing relation to compressibly engage the injured portion of an external ear. The pressure applying assembly includes an adjustment mechanism for forcing one pad assembly towards the other in a continuously adjustable, controlled manner creating a compression of the ear tissues required for proper healing between the two opposing pads.

This application is a continuation of U.S. patent application Ser. No. 11/544,721 filed on Oct. 10, 2006.

### BACKGROUND OF THE INVENTION

Devices and surgical procedures to aid in the prevention and healing of injuries to the external ear are rudimentary, crude, and painful. The most common such injury is auricular hematoma typically inflicted during contact sports such as wrestling, boxing, or rugby which if left untreated, or improperly treated, results in the hematomas becoming fibrotic and disfiguring, a thickening of the tissue known as “cauliflower ear”. Many protective devices such as U.S. Pat. Nos. 5,504,945 and 5,615,417 are intended to protect the tissues of the ear although history indicates devices such as these to either be too cumbersome to use consistently, minimally effective, or too inconvenient.

In addition to use in the treatment of auricular hematomas, pressure dressings are also used in many other treatments of the ear, such as the covering and compressing of an acute wound to the ear, the excision of a skin cancer, the placement of a skin graft, the repair of a torn earlobe, the treatment of a localized burn, or the excision of a keloid which can form on the earlobe or other portions of the ear after piercing thereof. Similar injuries and treatments occur with ears of animals.

Auricular hematomas are caused by a blunt trauma or shearing force to the external ear that disrupts the adherence of the perichondrium or skin of the ear to the underlying cartilage and the subsequent filling of the subperichondrial space with blood. Application of pressure when discomfort and preliminary trauma is incurred helps to prevent more serious damage. Once more serious damage has occurred, standard treatment involves needle aspiration of the hematoma or, better, incision and drainage, followed by compression of the injured area to prevent re-accumulation of fluid and to allow re-apposition of the perichondrium to the underlying cartilage; the application of pressure is crucial. Avoiding infection is another important measure to prevent further complications.

Several different compression techniques using pressure dressings have been employed to keep the skin in the necessary close contact with the cartilage during healing. The pressure dressings generally fall into the categories of suture compression dressings, mastoid dressings (i.e. dressings attached to the head by adhesive tape or other such methods), and molds (e.g. silicone) which are used with or without suturing or mastoid dressings.

Mastoid dressings are generally disfavored because of their bulkiness and tendency to come loose or be dislodged. Molds generally tend to be expensive and time consuming to apply. Since molds are very closely conforming, they do, however, tend to apply a more uniform pressure with force vectors that are directed orthogonally to a greater area of the structure involved in the injury. However, as the injured area becomes more or less inflamed either with the progression of healing and reduction of inflammation or by increased trauma and increase in inflammation, molds are unable to compensate for the adjustment in force required to maintain constant pressure.

Suture dressings, while invasive, are less bulky and more often effective. The most common method of applying pressure to the effected area involves suturing pressure dressing materials (often cotton balls or dental rolls with an antibiotic/antiseptic applied) positioned on opposing sides of the injured portion of the ear. The sutures are passed through the cartilage of the ear to gently squeeze the skin and cartilage together between the dressings. This method often does not provide evenly distributed pressure over the injured area and as a result, blood can re-accumulate under the skin to reform the hematoma. Reformation of the hematoma requires repeated aspiration of the accumulated blood. In order to provide a more evenly distributed pressure over the entire area of the injury, multiple sutures are necessary. Not only do these sutures through the ear cause much pain, but the risk of infection increases; with each aspiration or re-incision, infection potential is greater.

U.S. Pat. No. 5,827,212 attempts to more evenly distribute the pressure over the injured area although it still involves painful sutures through the ear tissue, introduces an increased risk of infection, and does not allow for adjustment of the applying force once installed.

U.S. Pat. No. 5,295,950 provides a non-invasive resolution of providing pressure to the outer ear utilizing a ductile metal strip with cushioning pads, eliminating the pain and risk of infection associated with penetrating the ear tissue with sutures. However, the force and resulting pressure applied to the ear is fixed by the physical nature of the ductile metal and amount of pressure applied when installed. No adjustability which is critical over time is included in this design other than removing and reapplying the device. Since the healing process takes several weeks for the skin to reattach, the dressing must be left in position for extended periods. As the healing process continues and the fluid increases or decreases, this method is unable to adjust for the change in thickness of the ear and pressure within the subperichondrial space. Loose dressings become less effective in maintaining sufficient pressure for complete heating and thickening and permanent deformity of the tissue can result.

A concept used for applying pressure on various other jointed body parts as in ankle, knee, neck or back splints and supports is a pneumatic concept and is utilized in U.S. Pat. Nos. 5,125,400, 5,316,547, 5,348,530, 5,407,421, 5,520,622, 5,542,911, and 5,623,723 although not specifically for ears

The present inventors recognize it would be advantageous to provide a non-invasive device which provides a constant, evenly applied pressure over the effected area of the injured ear, by being able to be easily adjusted upon initial application and over the course of the healing process of several weeks.

The present inventors recognize it would be advantageous to provide a device which reduces the pain associated with treatments currently used.

The present inventors recognize it would be advantageous to minimize the risk of infection associated with treatments commonly in use.

The present inventors recognize it would be advantageous to provide a reusable device.

The present inventors recognize it would be advantageous to provide a device that can adjust continuously within an operative range the pressure applied.

### SUMMARY OF THE INVENTION

The invention provides a non-invasive compression type device for aiding in the prevention, treatment and healing of injuries of the external ear (pinna). The device provides n continuously adjustable within an operative range and re-useable mechanism for continuously applying even pressure directly to the outer ear often needed in the treatment of auricular hematoma.

The device comprises a structure which extends over the cartilaginous rim portions of the ear or structures on either side of the external ear that allows application of pressure to the effected area is employed which incorporates pads or cushions. Application of pressure is accomplished by adjustably moving one of the pads closer towards the other creating a progressively smaller space between the pads within which the skin and tissue of the ear are caused to compress and re-adhere or remain intact; any fluids within the spaces of the perichondrium are forced out and cannot re-enter.

The device comprises an adjustment mechanism for creating the force for applying pressure. The adjustment device may be implemented in various embodiments and include mechanisms such as a screw, eccentric cam, inclined plane, fluidic, electromagnetic, and magnetic. These mechanisms can provide continuous adjustability within an operative range, allows re-use of the device if removed from the ear for any reason, and manually compensates for increases or reductions in pressure of the subperichondrial space in the effected area from increasing inflammation or decreasing amounts of fluid due to healing or other factors.

Numerous other advantages and features of the present invention will be become readily apparent from the following detailed description of the invention and the embodiments thereof, from the claims and from the accompanying drawings.

### BRIEF DESCRIPTION OF THE DRAWINGS

FIG. 1 is a side view showing an embodiment of the invention positioned on an ear;

FIG. 2 is a rear view taken from FIG. 1 with a screw thread pressure applying mechanism;

FIG. 3 is a rear view taken from FIG. 1, with a pressure applying mechanism located on the opposite (inside, close to the head) side of the ear as that shown in FIG. 2;

FIG. 4 is a section view of the embodiment shown in FIG. 2 taken along line 4-4 of FIG. 1;

FIG. 5 is a section view similar to FIG. 4 of an alternative embodiment having a screw thread pressure applying mechanism;

FIG. 6 is a section view similar to FIG. 4 of an alternative embodiment having a screw thread pressure applying mechanism;

FIG. 7 is a section view similar to FIG. 4 of an alternative embodiment having a screw thread pressure applying mechanism;

FIG. 8 is a section view similar to FIG. 4 of an alternative embodiment having an eccentric cam pressure applying mechanism;

FIG. 9 is a section view similar to FIG. 4 of an alternative embodiment having an inclined plane pressure applying mechanism;

FIG. 10 is a section view similar to FIG. 4 of an alternative embodiment having a fluidic (pneumatic) pressure applying mechanism;

FIG. 11 is a section view similar to FIG. 4 of an alternative embodiment having an electro-magnetic solenoid pressure applying mechanism;

FIG. 12 is a section view similar to FIG. 4 of an alternative embodiment having a magnetic pressure applying mechanism.

### DETAILED DESCRIPTION

While this invention is susceptible of embodiments in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

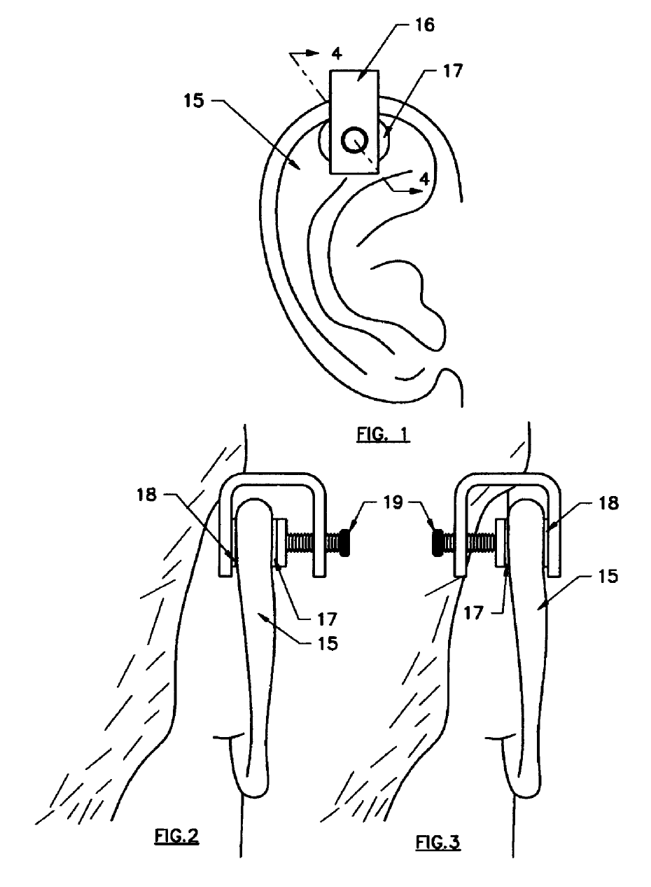

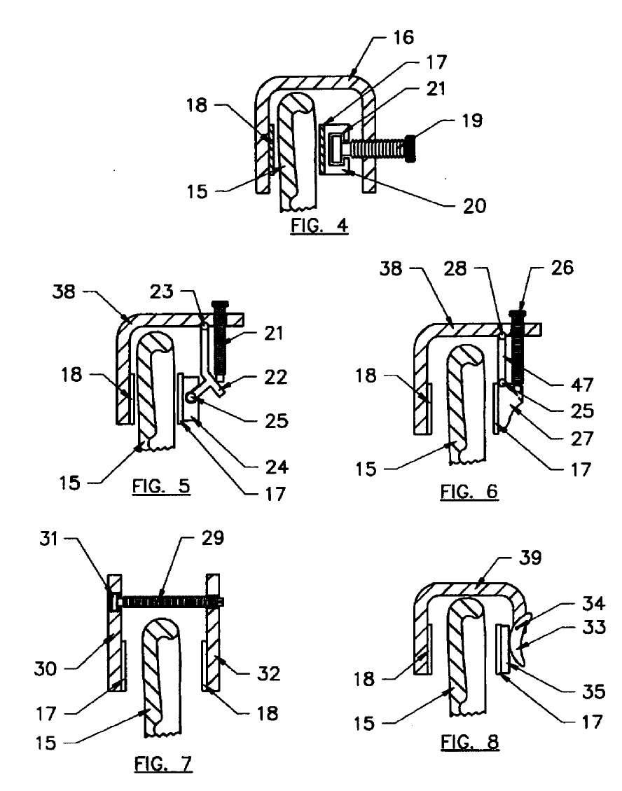

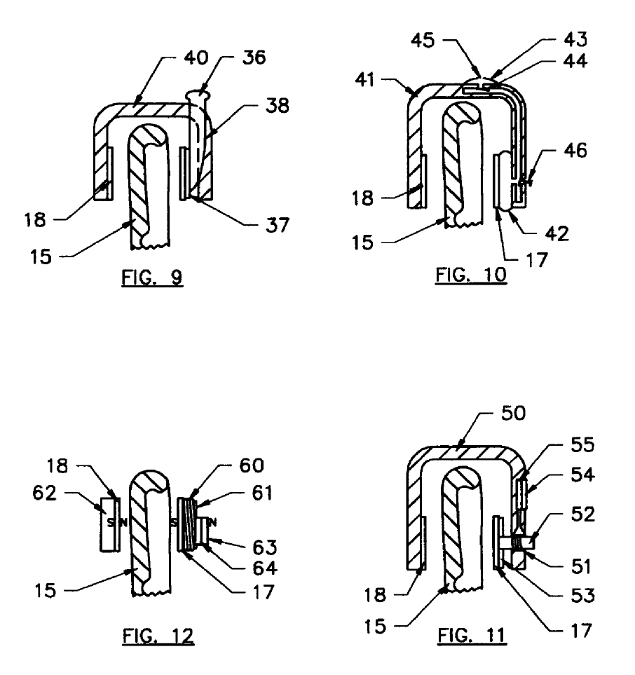

FIG. 1 shows a side view of the pinna 15 or outer ear with a structure 16 of one embodiment of the invention utilizing one example of a screw thread pressure applying mechanism. Pad 17 is shown on the outside surface of the pinna 15 in relative position depending on specific location of the hematoma and to assure adequate compression of the underlying tissues of the perichondrium.

FIG. 2 is a rear view of FIG. 1 with the screw thread pressure applying device located on the outer (away from the head) side of the pinna 15. As the screw thread 19 is adjusted so as to reduce the space between pads 17 and 18, the underlying tissues of the perichondrium are compressed between said pads. In an embodiment, the pads 17, 18 comprise gelatinous fluid or other materials conformable to the contours of the pinna when engaged with the pinna. The gelatinous fluid or said other materials may be resilient to return the pad to its initial, nonconformed, condition when not engaged with the ear.

FIG. 3 is a rear view of FIG. 1 with the screw thread pressure applying mechanism located on the inner (close to the head) side of the pinna 15. As the screw thread 19 is adjusted so as to reduce the space between pads 17 and 18, the underlying tissues of the perichondrium are compressed between said pads. The operation of the pressure applying device is acting in the same manner as in FIG. 2 only from this alternate position.

FIG. 4 is a section view of the screw thread pressure applying device of FIG. 2 showing the attachment detail of the threaded screw 19 to the actuating pad 17 holder 20. The rotational movement of the threaded screw 19 is free with respect to the pad 17 holder 20 as the end of the threaded screw 19 is captured loosely within a cavity 21 in pad 17 holder 20 such that the pad 17 holder 20 is pushed or pulled as moves the threaded screw 19, thus allowing the pad 17 to maintain its fixed position on the pinna 15 while the inward or outward movement of the threaded screw relative to the structure 16 creates the force which is transmitted directly to the pad 17. This force creates an evenly applied pressure across the face of said pad 17 to the underlying tissues of the perichondrium and is equally resisted on the opposite side of the pinna 15 by the pad 18.

FIG. 5 is a section view similar to FIG. 4 of an alternative embodiment having a structure 38 utilizing a second example of a threaded screw pressure applying device. As the rotational movement of the threaded screw 21 moves vertically downward, contact is made with the connecting arm 22 hinged at 23. The force is transmitted through this connecting arm 22 to the actuating pad 17 holder 24 via the pivoting joint 25 whereby the pad 17 is free to maintain its fixed position against the pinna 15. This creates an evenly applied pressure across the face of said pad 17 to the underlying tissues of the perichondrium which is equally resisted on the opposite side of the pinna 15 by the pad 18 via the structure 38. As noted in FIG. 3, the pressure applying device may also be located on the inner side of the pinna 15.

FIG. 6 is a section view similar to FIG. 4 of an alternative embodiment having a structure 38 utilizing a third example of a threaded screw pressure applying device. As the rotational movement of the threaded screw 26 moves vertically downward, contact is made with the angled pad 17 holder 27 hinged at 28 with a connecting swing arm 47 hinged at 25. The force is transmitted through this angled pad 17 holder 27 to the actuating pad 17 such that the pad 17 is free to maintain its fixed position against the pinna 15 while the movement creates an evenly applied pressure across the face of said pad 17 to the underlying tissues of the perichondrium and is equally resisted on the opposite side of the pinna 15 by the pad 18 via the structure 38. As noted in FIG. 3, the pressure applying device may also be located on the inner side of the pinna 15.

FIG. 7 is a section view similar to FIG. 4 of an alternative embodiment having a fourth example of a threaded screw pressure applying device. The rotational movement of the threaded screw 29 is free with respect to the pad 17 holder 30 as the end of the threaded screw 29 is captured loosely within a cavity 31 in pad 17 holder 30 such that the pad 17 holder 30 is pushed or pulled as moves the threaded screw 29 in corresponding threads in pad 18 holder 32 thus allowing the pad 18 to maintain its fixed position on the pinna 15 while the inward or outward movement of the threaded screw 29 relative to pad 18 holder 32 creates the force which is transmitted directly to the pad 18. This force creates an evenly applied pressure across the face of said pad 18 to the underlying tissues of the perichondrium and is equally resisted on the opposite side of the pinna 15 by the pad 17. As noted in FIG. 3, the pressure applying device may also be located on the inner side of the pinna 15.

FIG. 8 is a section view similar to FIG. 4 of an alternative embodiment having a structure 39 utilizing an eccentric cam lever 33 as the pressure applying device. As eccentric cam lever 33 rotates about pivot point 34, the increasing or decreasing radius at the point of contact with pad 17 holder 35 causes pad 17 holder 35 and pad 17 to move inward or outward relative to pinna 15. The force is transmitted through this pad 17 holder 35 to the pad 17 such that the pad 17 is free to maintain its fixed position against the pinna 15 while the movement creates an evenly applied pressure across the face of said pad 17 to the underlying tissues of the perichondrium and is equally resisted on the opposite side of the pinna 15 by the pad 18 via the structure 39. As noted in FIG. 3, the pressure applying device may also be located on the inner side of the pinna 15.

FIG. 9 is a section view similar to FIG. 4 of an alternative embodiment having a structure 40 utilizing an inclined plane 36 as the pressure applying device. As the inclined plane 36 is pushed vertically downward within slot 38 in structure 40 contact is made with the pad 17 holder 37 causing pad 17 holder 37 and pad 17 to move inward or outward relative to pinna 15. The force is transmitted through pad 17 holder 37 to the pad 17 such that pad 17 and pad 17 holder 37 is free to maintain its fixed position against the pinna 15 while the movement creates an evenly app lied pressure across the face of said pad 17 to the underlying tissues of the perichondrium and is equally resisted on the opposite side of the pinna 15 by the pad 18 via the structure 40. As noted in FIG. 3, the pressure applying device may also be located on the inner side of the pinna 15.

FIG. 10 is a section view similar to FIG. 4 of an alternative embodiment having a structure 41 incorporating a fluidic pressure applying device. In this view, a pneumatic bladder 42 fills with air and expands thus causing pad 17 attached to pneumatic bladder 42 to move inward or outward relative to pinna 15. The force is transmitted through pad 17 such that the movement creates an evenly applied pressure across the face of said pad 17 to the underlying tissues of the perichondrium and is equally resisted on the opposite side of the pinna 15 by the pad 18 via the structure 41. As noted in FIG. 3, the pressure applying device may also be located on the inner side of the pinna 15.

Pressure is created by pressing on the flexible bulb 43 with the pressing member, usually a person's finger, covering the hole 45 with which air initially fills the space within the bulb. This pressing action and resulting collapse of the bulb causes the entrapped air to be pushed into the internal cavities of structure 41 through a pressure retention normally closed check valve 44 and into the pneumatic bladder 42. Pressure can be manually released from the internal cavities of structure 41 and pneumatic bladder 42 by pressing pressure retention normally closed valve 46.

FIG. 11 is a section view similar to FIG. 4 of an alternative embodiment having a structure 50 incorporating an electromagnetic solenoid pressure applying device. In this view, an electric solenoid coil 51 surrounds solenoid plunger 52 which is integrally attached to pad 17 holder 53. Electrically connected to one end of the electric solenoid coil 51 wire is an electrical circuit including a variable resistance capability, electricity flow direction control, and on/off switching capability 55 which connects to the positive side of disk battery 54. The other wire of the electric solenoid coil 51 is connected electrically to the negative side of disk battery 54. As electricity is applied to electric solenoid coil 51 from disk battery 54 the electromagnetic field created causes the solenoid plunger 52 to move inward or outward relative to, pinna 15. The movement is transmitted through pad 17 such that the force creates an evenly applied pressure across the face of said pad 17 to the underlying tissues of the perichondrium and is equally resisted on the opposite side of the pinna 15 by the pad 18 via the structure 50. As noted in FIG. 3, the pressure applying device may also be located on the inner side of the pinna 15.

FIG. 12 is a section view similar to FIG. 4 of an alternative embodiment having a magnetic pressure applying device. In this view, an electric coil 60 surrounds permanent magnet 61 which is integrally attached to pad 17. Pad 18 is integrally attached to permanent magnet 62. Each permanent magnet 61 and 62 have north-south poles as indicated, and are oriented such that the opposing poles attract each other. The magnetic forces thus created cause an evenly applied pressure across the face of pad 17 to the underlying tissues of the perichondrium and is equally resisted on the opposite side of the pinna 15 by the pad 18.

Electrically connected to one end of the electric coil 61 wire is an electrical circuit including a variable resistance capability, electricity flow direction control, and on/off switching capability 64 which connects to the positive side of disk battery 63. The other wire of the electric coil 61 is connected electrically to the negative side of disk battery 63. As electricity is applied to electric coil 61 from disk battery 63 the electromagnetic field, created causes the strength of permanent magnet to be reduced or increased depending on flow of electricity direction thereby allowing this electrical circuitry to provide adjustability of the magnetic attraction and resulting pressure transmitted across the face of pad 17 to the underlying tissues of the perichondrium which is equally resisted on the opposite side of the pinna 15 by the pad 18. As noted in FIG. 3, the pressure applying device may also be located on the inner side of the pinna 15.

From the foregoing, it will be observed that numerous variations and modifications may be effected without departing from the spirit and scope of the invention. In actual production designs, provisions for user friendliness, for economic manufacturing practices and techniques, and for maximizing effectiveness would be included and provided for which have not been identified as part of this patent but which would still render the device as being completely covered under the intent of this patent. The embodiment previously described is illustrative of the principles of this invention. It should be understood, modifications can be made without departing from the scope of the invention. It is to be understood that no limitation with respect to the specific apparatus illustrated herein is intended or should be inferred. It is intended to cover by the appended claims all such modifications as fall within the scope of the claims.

The invention claimed is:

1. A pinna compression device for treating injuries to the pinna, comprising:
a support operatively connecting a first contact area to an oppositely facing second contact area, the first and second contact areas for engaging the pinna;
the support extending between the first and second contact areas in an area outside of a compression space, the compression space comprises the area between the first and second contact areas;
the support comprises an adjustment mechanism operatively connected to at least one of the first and second contact areas,
the adjustment mechanism moves at least one of the first or second contact areas toward the other of the first or second contact areas to compress the pinna between the first and second contact areas; and
the first and second contact areas each comprising conformable pressure pads conformingly engagable with the surface of the pinna to apply substantially uniform pressure from the first and second contact areas to the pinna between the first and second contact areas when the adjustment mechanism is in an engaged position;
the adjustment mechanism is variable in positioning at least one of the first or second contact areas with respect to the other of the first or second contact areas to vary the amount of pressure applied to the pinna between the first and second contact areas.

2. The device of claim 1, wherein each of the first and second contact areas comprise a hard part supporting a soft part where the soft part of the first contact area faces the soft part of the second contact area.

3. The device of claim 1, wherein the first contact area has a first contact surface area and the second contact area has a second contact surface area; the first surface area is substantially equal to the second surface area.

4. The device of claim 1, wherein the support is ridged for transferring force created by the adjustment mechanism evenly across the first and second contact areas; the support in a substantially unflexed condition when the adjustment mechanism is in the engaged position.

5. The device of claim 1, wherein the first and second contact areas are substantially flat.

6. The device of claim 1, wherein the adjustment mechanism is continuously variable within an operable range in positioning the at least one of the first or second contact areas with respect to the other of the first or second contact areas to vary the amount of pressure applied the pinna between the first and second contact areas when the adjustment mechanism is in an engaged position.

7. The device of claim 1, wherein the adjustment mechanism comprises a screw threaded through a portion of the support, one end of the screw is operatively connected to one of the first or second contact areas to move at least one of the first or second contact areas toward the other of the first or second contact areas.

8. The device of claim 7, wherein the screw is substantially perpendicular to one of the first or second contact areas.

9. The device of claim 1, wherein the adjustment mechanism comprises a screw threaded through a portion of the support, and a connecting arm pivotably connected to a portion of the support at a first end and pivotably connected to one of the first or second contact areas at an opposite end;
the screw is positioned to contact a portion of the connecting arm to move one of the first or second contact areas toward the other of the first or second contact areas.

10. The device of claim 1, wherein the support comprises a first part, a second part, and a screw connecting the first part to the second part, and wherein the adjustment mechanism comprises the screw;
the first part comprises the first contact area;
the second part comprises the second contact area; and
the screw is threaded into one of the first or second parts to move at least one of the first or second contact areas toward the other of the first or second contact areas.

11. The device of claim 1, wherein the support comprises a first part, a second part; the first part comprises the first contact area, and the second part comprises the second contact area; and
the adjustment mechanism comprises a lever cam and a pivot point; the cam lever is operatively connected about the pivot point to one of the first or second parts,
the cam contacts one of the first or second contact areas corresponding to the first or second part that is operatively connected at the pivot point;
the cam is shaped to create an increasing radius about the pivot point when the cam is pivoted in a first direction for moving said first or second contact area toward the other of the other of the first or second contact areas.

12. The device of claim 1, wherein the adjustment mechanism comprises a lever and slot formed in the support;
one of the first or second contact areas is operatively connected to one end portion of the lever,
the slot comprises a wall portion facing the other of the first or second contact areas,
a lower portion of the wall portion is vertically closer to the other of the first or second contact areas than an upper portion of the wall portion,
the lever is movable within the slot and the lower portion directs the lever and the first or second contact area toward the other of the first or second contact areas when the adjustment mechanism is moved toward the engaged position.

13. The device to claim 1, wherein the adjustment mechanism comprises a fluidic device operatively connected to the support and to at least one of the first or second contact areas, the fluidic device moves said first or second contact area toward the other of the first or second contact areas when fluid or air is supplied to the fluidic device.

14. The device of claim 1, wherein the adjustment mechanism comprises a solenoid connected to the support, a movable element of the solenoid is operatively connected to one of the first or second contact areas to move the first or second contact area toward the other of the first or second contact areas.

15. The device of claim 1, wherein the adjustment mechanism comprises a magnetic device;
the magnetic device comprises a electric coil surrounding a first permanent magnet attached to one of the first or second contact areas, and a second permanent magnet attached to the other of the first or second contact areas, a pole of one magnet facing an opposite pole of the other magnet.

16. An ear compression device for treating injuries to the ear, comprising:
a support for connecting a first contact area to an oppositely facing second contact area, the first and second contact areas for engaging the pinna;
a first means operatively connected to at least one of the first contact area or second contact area for moving at least one of the first or second contact areas toward the other of the first or second contact areas to compress the ear between the first and second contact areas; and
the first means also for varying the position of at least one of the first or second contact areas with respect to the other of the first or second contact areas to vary the amount of pressure applied the ear between the first and second contact areas; and
a surface contour of the first contact area is substantially the same as a surface contour of the second contact area when the first means is in an unengaged position wherein at least one of the first or second contact areas are unengaged with the ear.

17. The compression device of claim 16, wherein the support operatively connects the first contact area to the oppositely facing second contact area in a manner physically non-invasive to the ear; and
the first and second contact areas each comprising conformable pressure pads conformingly engagable with the surface of the pinna to apply substantially uniform pressure from the first and second contact areas to the pinna between the first and second contact areas when the adjustment mechanism is in an engaged position.

18. A method of treating injuries to the pinna, comprising
providing a first contact area and an oppositely facing second contact area;
providing a support structure operatively connecting the first and second contact areas; and
applying substantially uniformly pressure from the first and second contact areas to the pinna between the first and second contact areas wherein the support structure is physically non-invasive to the pinna.

19. The method of claim 18, wherein the step of applying includes the step of
conforming the first and second contact areas with the surface of the pinna by engaging the first and second contract areas with the pinna, wherein the first and second contact areas comprise conformable pressure pads.

20. The method of claim 18, where the step of applying comprises the step of adjusting the pressure applied through a continuously variable adjustment mechanism that is continuously variable within an operative range and operatively connected to at least one of the first and second contact areas.
